(19) United States
(12) Patent Application Publication
(10) Pub. No.: US 2021/0163567 A1
(43) Pub. Date: Jun. 3, 2021

(54) Method of Delivering a Centrally-Acting Peptide Derivative, and Method of Treating a Neuropsychiatric Disorder

(71) Applicant: TOKYO UNIVERSITY OF SCIENCE FOUNDATION, Tokyo (JP)

(72) Inventors: Chikamasa Yamashita, Tokyo (JP); Jun-Ichiro Oka, Tokyo (JP); Michiko Horiguchi, Tokyo (JP); Sachie Sasaki-Hamada, Tokyo (JP)

(21) Appl. No.: 17/172,408

(22) Filed: Feb. 10, 2021

Related U.S. Application Data
(62) Division of application No. 15/507,403, filed on Feb. 28, 2017.

(30) Foreign Application Priority Data
Sep. 2, 2014 (JP) 2014-177974
Sep. 10, 2014 (JP) 2014-184436

(51) Int. Cl. C07K 14/605; A61K 38/26; C07K 19/00; A61K 38/22; A61K 38/28; A61K 38/095; A61K 9/00

(52) U.S. Cl. CPC C07K 14/605; A61K 38/26; C07K 19/00; A61K 38/2271; A61K 38/00; A61K 38/28; A61K 38/095; A61K 9/0043; A61K 38/2264

(57) ABSTRACT

A method of delivering a centrally-acting peptide derivative, and method of treating a neuropsychiatric disorder.

### CROSS-REFERENCE TO RELATED APPLICATIONS

This application is a divisional of U.S. Application No. 15/507,403 submitted Feb. 28, 2017, which is the U.S. National Stage of International Application No. PCT/JP2015/074962 filed Sep. 2, 2015, which claims the priority benefit of JP Application No. 2014-177974 filed Sep. 2, 2014, and JP Application No. 2014-184436 filed Sep. 10, 2014, the entire respective disclosures of which are incorporated herein by reference.

### TECHNICAL FIELD

The invention relates to a centrally-acting peptide derivative and a pharmaceutical composition.

### BACKGROUND ART

In recent years, there has been a large amount of research on the pharmacological applications of the physiological activities of substances, such as peptides, that are derived from biological bodies. For example, it is known that the glucagon-like peptide-1 (GLP-1), which is a peptide that is derived from proglucagon and formed from 37 amino acid residues, and the glucagon-like peptide-2 (GLP-2), which is a peptide that is derived from proglucagon and formed from 33 amino acid residues, bind to a G-protein-coupled receptor (GPCR) and activate signaling transduction.

Regarding the pharmacological activity of GLP-1 (amide activity types 7-37 and 7-36 are present) in the brain, there have been reports of an effect of alleviating learning disorders (for example, Non-Patent Documents 1 and 2).

Regarding the pharmacological activity of GLP-2 in the brain, there have been reports of effects of anti-depresssive activity even in treatment resistant depression model animals, lowering blood pressure and alleviating learning disorders (for example, Non-Patent Documents 3 to 9).

Further, it has been reported that neuromedin U (NmU), which is a peptide formed of 23 amino acid residues, binds to a GPCR in the brain and exerts an effect of alleviating learning disorders (for example, Non-Patent Document 10).

Peptides having a central activity such as opioid peptides, oxytocin, leptin, neuropeptide Y, orexin and insulin, and antibody drugs, have also been developed.

### PRIOR ART DOCUMENT

### Non-Patent Document

Non-Patent Document 1: Neuroscience Research 64(2009)67-74

Non-Patent Document 2: Journal of Neuroscience Research 92(2014)446-454

Non-Patent Document 3: Behavioural Brain Research 204(2009)235-240

Non-Patent Document 4: Neuroscience 212(2012)140-148

Non-Patent Document 5: Life Sciences 93(2013)889-896

Non-Patent Document 6: Neuroscience Letters 550(2013)104-108

Non-Patent Document 7: Behavioural Brain Research 243(2013)153-157

Non-Patent Document 8: Neuropeptides 49(2015)7-14

Non-Patent Document 9: Neuroscience 294(2015)156-165

Non-Patent Document 10: Neuroscience Research 61(2008)113-119

### SUMMARY OF THE INVENTION

### Problem to be Solved by the Invention

It is anticipated that substances derived from a biological body, such as peptides, may be used as an active component of a drug with fewer side effects than previous drugs. It is desirable for drugs that need to be taken on a daily basis to be administered in a less invasive manner.

Therefore, development of a nasal formulation as a dosage form that is adaptable for less-invasive administration has been studied. However, there have been problems in that a peptide that has been administered in an intranasal manner does not easily pass the blood-brain barrier, or that the effect of a peptide is lost within a short period of time due to enzymatic decomposition in a biological body.

The inventors have focused on intranasal administration, which is a promising administration in terms of being less invasive and capable of delivering a peptide to the central nervous system, and have enabled the delivery of a centrally-acting peptide to the brain in an efficient manner by adding a peptide having a specific sequence to the centrally-acting peptide.

Specifically, the invention aims to provide a centrally-acting peptide derivative having a superior ability to migrate to the central nervous system, and a pharmaceutical composition including the centrally-acting peptide derivative.

### Means for Solving the Problem

The means for solving the problem includes the following embodiments.

<1> A centrally-acting peptide derivative, comprising a centrally-acting portion, a cell-penetrating sequence portion, and an endosomal-escape portion.

<2> The centrally-acting peptide derivative according to <1>, wherein the centrally-acting portion is an amino acid sequence portion that is derived from a peptide.

<3> The centrally-acting peptide derivative according to <2>, wherein the amino acid sequence portion is derived from a peptide that is GLP-1, GLP-2, neuromedin U, an opioid peptide, oxytocin, leptin, orexin, neuropeptide Y or insulin.

<4> The centrally-acting peptide derivative according to <2> or <3>, wherein the amino acid sequence portion is derived from a peptide selected from the following (a1), (a2), (a3) or (b):

(sequence number 1)
(a1) a peptide comprising an amino acid sequence
represented by His-Ala-Asp-Gly-Ser-Phe-Ser-Asp-
Glu-Met-Asn-Thr-Ile-Leu-Asp-Asn-Leu-Ala-Ala-Arg-
Asp-Phe-Ile-Asn-Trp-Leu-Ile-Gln-Thr-Lys-Ile-Thr-
Asp;
(sequence number 2)
(a2) a peptide comprising an amino acid sequences
represented by His-Ala-Glu-Gly-Thr-Phe-Thr-Ser-
Asp-Val-Ser-Ser-Tyr-Leu-Glu-Gly-Gln-Ala-Ala-Lys-
Glu-Phe-Ile-Ala-Trp-Leu-Val-Lys-Gly-Arg-NH₂;
(neuromedin U, sequence number 3)
(a3) a peptide comprising an amino acid sequence
represented by Tyr-Lys-Val-Asn-Glu-Tyr-Gln-Gly-
Pro-Val-Ala-Pro-Ser-Gly-Gly-Phe-Phe-Leu-Phe-Arg-
Pro-Arg-Asn-NH₂;

and

(b) a peptide comprising an amino acid sequence represented by any of (a1) to (a3) in which one or more amino acid residues are deleted, replaced or added, the peptide having a centrally-acting activity.

<5> The centrally-acting peptide derivative according to any one of <1> to <4>, used for treatment of a neuropsychiatric disorder.

<6> The centrally-acting peptide derivative according to any one of <1> to <4>, used for treatment of depression or a learning disorder.

<7> A pharmaceutical composition, comprising the centrally-acting peptide derivative according to any one of claim 1 to <6>as an active ingredient.

<8> The pharmaceutical composition according to <7>, used for treatment of a neuropsychiatric disorder.

<9> The pharmaceutical composition according to <7> or <8>, used for treatment of depression or a learning disorder.

<10> A nasal formulation comprising the centrally-acting peptide derivative according to any one of <1> to <6> as an active ingredient.

<11> The nasal formulation according to <10>, used for treatment of a neuropsychiatric disorder.

<12> The nasal formulation according to <10> or <11>, used for treatment of depression or a learning disorder.

<13> Use of a pharmaceutical composition for intranasal administration, the pharmaceutical composition comprising the centrally-acting peptide derivative according to any one of <1> to <6> as an active ingredient.

<14> A method of stabilizing a centrally-acting peptide, the method comprising adding a cell-penetrating sequence portion and an endosomal-escape portion to a centrally-acting peptide.

### Effect of the Invention

According to the invention, it is possible to provide a centrally-acting peptide derivative having a superior ability to migrate to the central nervous system, and a pharmaceutical composition including the centrally-acting peptide derivative.

### BRIEF EXPLANATION OF THE DRAWINGS

FIG. 1 shows the result of a forced-swim test on a rat administered with a GLP-2 derivative.

FIG. 2 shows the result of a forced-swim test on a rat administered with GLP-2 alone.

FIG. 3 shows the distribution of a GLP-2 derivative in the brain of a rat.

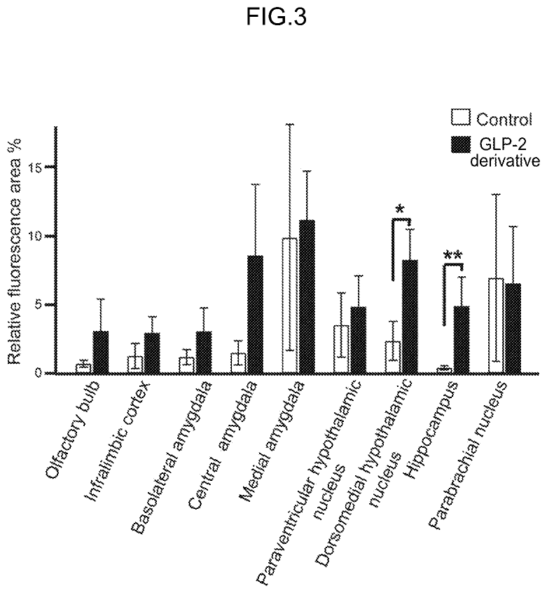

FIG. 4 shows the result of a forced-swim test on a mouse administered with a GLP-2 derivative.

FIG. 5 shows the result of a tail suspension test on a mouse administered with a GLP-2 derivative.

FIG. 6 shows the result of a forced-swim test on a treatment-resistant depression model mouse administered with a GLP-2 derivative.

FIG. 7 shows the result of a Y-maze test on a learning disorder model mouse administered with a GLP-1 derivative.

FIG. 8 shows the result of a Y-maze test on a learning disorder model mouse administered with an NmU derivative.

FIG. 9 shows the result of a stability test of DPP-4, which is an enzyme that decomposes a GLP-2 derivative.

### EMBODIMENTS FOR IMPLEMENTING THE INVENTION

In the following, embodiments of the invention are explained. The explanation and the embodiments are exemplify the invention, and do not limit the scope of the invention.

Numerical ranges indicated as “from A to B” described herein include A and B as a minimum value and a maximum value, respectively.

The left side of the amino acid sequence refers to the N-terminal, and the amino acid residue may be indicated by a one-letter abbreviation (for example, G for glycine residue) or a three-letter abbreviation (for example, Gly for glycine residue).

“Treatment” as described herein refers not only to an activity or an effect that quenches or alleviates a symptom, but also to an activity or an effect that suppresses aggravation of the symptom.

An “anti-depressive activity” or “anti-depressive effect” refers not only to an activity or an effect that quenches a symptom of depression, but also to an activity or an effect that suppresses aggravation of the symptom.

A “learning disorder-alleviating activity” or “learning disorder-alleviating effect” refers not only to an activity or an effect that quenches a symptom of a learning disorder, but also to an activity or an effect that suppresses aggravation of the symptom.

### <Centrally-acting Peptide Derivative>

The centrally-acting peptide derivative of the invention has a centrally-active portion, a cell-penetrating sequence portion and an endosomal-escape portion. The inventors have studied on a method to deliver a centrally-acting peptide to the brain in an expeditious manner before the peptide decomposes in a body, and to deliver a centrally-acting peptide to the brain in an efficient manner. As a result, the inventors have found that the ability of a peptide to migrate to a brain is significantly improved when the peptide is intranasally administered as derivative having a centrally-active portion, a cell-penetrating sequence portion and an endosomal-escape portion, as compared with the case in which the centrally-active portion alone is intranasally administered.

The intended purpose of the centrally-acting peptide derivative is not limited, as long as it employs a pharmacological activity that is expressed by the centrally-acting portion upon acting on the central nervous system. Examples of the pharmacological activity include an anti-depression activity, a learning disorder-alleviating activity, an anti-anxiety activity, a feeding suppression activity, a cognitive disorder-alleviating activity, a blood-pressure-lowering activity, an analgesic activity, a sleep-inducing activity, and an anti-epileptic activity. Therefore, the centrally-acting peptide derivative is suitably used as a drug for the treatment of neuropsychiatric disorders, such as an anti-depression agent, a learning disorder-alleviating agent, an anti-anxiety agent, a feeding suppression agent, a cognitive disorder-alleviating agent, a blood-pressure-lowering agent, an analgesic agent, a sleep-inducing agent, and an anti-epileptic agent.

### (Centrally-acting Portion)

The centrally-acting portion in the centrally-acting peptide derivative is not particularly limited, as long as it exerts a pharmacological activity by acting on the central nervous system. Addition of a cell-penetrating sequence portion and an endosomal-escape portion to a centrally-acting portion may be performed by a known process without particular restriction.

In an embodiment of the centrally-acting peptide derivative, the centrally-acting portion is an amino acid sequence portion derived from a centrally-acting peptide (also referred to as a centrally-acting peptide portion). Examples of the centrally-acting peptide include GLP-1, GLP-2, neuromedin U, opioid peptide (such as enkephalin and dynorphin), oxytocin, leptin, orexin, neuropeptide Y and insulin.

In an embodiment of the centrally-acting peptide derivative, the centrally-acting portion is a peptide portion derived from a peptide that is any of the following (a1) to (a3) or (b).

(GLP-2, sequence number 1)
(a1) a peptide comprising an amino acid sequence
represented by HADGSFSDEMNTILDNLAARDFINWLIQTKITD
(GLP-1, 7-36 amide, sequence number 2)
(a2) a peptide comprising an amino acid
sequences represented by HAEGTFTSDVSSYLEGQAAKEFIAW
LVKGR-NH₂
(neuromedin U, sequence number 3)
(a3) a peptide comprising an amino acid sequence
represented by YKVNEYQGPVAPSGGFFLFRPRN-NH₂

(b) a peptide comprising an amino acid sequence represented by any of (a1) to (a3) in which one or more amino acid residues are deleted, replaced or added, the peptide having a centrally-acting activity.

Among these peptides, GLP-2 exerts an anti-depression activity and a blood-pressure-lowering activity, GLP-1 exerts a learning disorder-alleviating activity, and neuromedin U exerts a learning disorder-alleviating activity. Therefore, these peptides are useful as a centrally-acting portion of the centrally-acting peptide derivative.

When a peptide is formed of an amino acid sequence of a specific peptide and an amino acid sequence of a different peptide, the term “amino acid sequence derived from the specific peptide” refers to a portion of the peptide corresponding to the amino acid sequence of the specific peptide.

When the centrally-acting portion is derived from “(b) a peptide including an amino acid sequence represented by any of (a1) to (a3) in which one or more amino acid residues are deleted, replaced or added”, the number of the amino acid residues to be deleted, replaced or added is not particularly limited as long as the centrally-acting peptide portion exert the effect of the invention. For example, the number of the amino acid residues to be deleted, replaced or added may be from 1 to 10, preferably from 1 to 5, more preferably from 1 to 3. From the viewpoint of synthetic efficiency, handleability, stability and the like, the total number of the amino acid residues of the peptide (b) is preferably 40 or less, more preferably 35 or less, further preferably 33 or less.

### (Cell-penetrating Sequence Portion)

The centrally-acting peptide derivative has a cell-penetrating sequence portion. The cell-penetrating sequence portion is formed of an amino acid sequence of a cell-penetrating peptide (CPP). The cell-penetrating peptide refers to a peptide having an activity to penetrate a cell membrane. The cell-penetrating sequence portion introduces the centrally-acting peptide derivative into a cell by its cell-penetrating activity. It is considered that the centrally-acting peptide derivative is delivered to the brain via nasal membrane, olfactory epithelium or the like, mainly through the axonal transport or the like, and is distributed therein.

The structure or the mechanism of penetration of a cell-penetrating peptide that constitutes the cell-penetrating sequence portion is not particularly limited, as long as it has an activity to introduce the centrally-acting peptide derivative into a cell.

Examples of the cell-penetrating peptide include oligoarginine (Rn, n is the number of arginine residues ranging from 6 to 12), Angiopep-5(RFFYGGSRGKRNNFRTEEY, sequence number 4), Antp (RQIKIWFQNRRMKWKK, sequence number 5), Bac (YGRKKRRQRRR, sequence number 6), BR1 (RAGLQFPVGRLLR, sequence number 7), BR2 (RAGLQFPVGRLLRRLLR, sequence number 8), Buf IIb[BR3] (RAGLQFPVGRLLRRLLRRLLR, sequence number 9), SH-CPPP-2 (KLPVM, sequence number 10), CyLoP-1(CRWRWKCCKK, sequence number 11), Cys-Antp (CRQIKIWFQNRRMKWKK, sequence number 12), Cys-pVEC (CLLIILRRRIRKQAHAHKS, sequence number 13), Cys-SAP (CVRLPPPVRLPPPVRLPPP, sequence number 14), Cys-SAPr (CPPPLRVPPPLRVPPPLRV, sequence number 15), Cys-TAT (CYGRKKRRQRRR, sequence number 16), Cys-TP10 (CAGYLLGKINLKALAALAKKIL, sequence number 17), Cys-TP1OK (CAGYLLGKINKLKALAALAKKIL, sequence number 18), FAK (FAKLAARLYRKALARQLGVAA, sequence number 19), H9(HHHHHHHHH, sequence number 20), HC-[poly(K)] (QYIKANSKFIGITEL-poly(K), sequence number 21), hLF (KCFQWQRNMRKVRGPPVSCIKR, sequence number 22), HR9 (CHHHHHHRRRRRRRRRHHHHHC, sequence number 23), IR9 (GLFEAIEGFIENGWEGMIDGWYGRRRRRRRRR, sequence number 24), JNKI-1 D-TAT (TDQSRPVQPFLNLTTPRKPRRPPRRRQRRKKRG, sequence number 25), K9(KKKKKKKKK, sequence number 26), low molecular weight protamine (LMWP) (VSRRRRRRGGRRRR, sequence number 27), MAP(KLALKLALKALKAALKLA, sequence number 28), Mitoparan (INLKKLAKL(Aib)KKIL, sequence number 29), m-N2-10-1(MTS-C) (HYSRLEICNL-AAVALLPAVLLALLP, sequence number 30), m-N2-10-1(MTS-N) (AAVALLPAVLLALLP-HYSRLEICNL, sequence number 31), m-N2-10-1 (NIV-C) (HYSRLEICNL-GRKKRRQRRRPPQ, sequence number 32), m-N2-10-1 (NIV-N) (GRKKRRQRRRPPQ-HYSRLEICNL, sequence number 33), m-N2-10-1 (OR-C) (RRRRRRRR-HYSRLEICNL, sequence number 34), m-N2-10-1 (OR-N) (HYSRLEICNL-RRRRRRRR, sequence number 35), MPG-8 (AFLGWLGAWGTMGWSPKKKRK-Cysteamide, sequence number 36), MPGα (GALFLAAALSLMGLWSQPKKKRKV-NH-CH2-CH2-SH, sequence number 37), MPGβ (GALFLGAALSLMGLWSQPKKKRKV-NH-CH2-CH2-SH, sequence number 38), NrTP1 (YKQCHKKGGKKGSG, sequence number 39), NrTP2 (YKQCHKKGG-Ahx-KKGSG, sequence number 40), NrTP5 (YKGCHKKGGKKGSG, sequence number 41), NrTP6 (YKQSHKKGGKKGSG, sequence number 42), NrTP7 (YRQSHRRGGRRGSG, sequence number 43), MPG HIV-gp41/SV40 T-antigen (GALFLGFLGAAGSTMGAWSQPKKKRKV, sequence number 44), NrTP8(WKQSHKKGGKKGSG, sequence number 45), PAMAM-PEG-Angiopep-2 (PFFYGGSGGNRNNYLREEY, sequence number 46), PEI-TAT (TGRKKRRQRRR-PEI, sequence number 47), penetratin (43-58) (RQIKIWFQNRRMKWKK, sequence number 48), penetratin (RQIKIWFQNRRMKWKKK, sequence number 49), Pep-1 HIV-reverse transcriptase/SV40 T-antigen (KETWWETWWTEWSQPKKKRKV, sequence number 50), Pep1 (KETWWETWWTEWSQPKKKRKV, sequence number 51), pepM residues 45-72 of DENV-2 C protein (KLFMALVAFLRFLTIPPTAGILKRWGTI, sequence number 52), pepR residues 67-100 of DENV-2 C protein (LKRWGTIKKSKAINVLRGFRKEIGRMLNILNRRRR, sequence number 53), peptide derived from protamine (RRRRRRGGRRRRG, sequence number 54), peptide vascular endothelial cadherin (pVEC) (LLIILRRRIRKQAHAHSK, sequence number 55), PF14 (AGYLLGKLLOOLAAAALOOLL, O represents any amino acid residue, sequence number 56), PR9 (FFLIPKGRRRRRRRRR, sequence number 57), protamine (VSRRRRRRGGRRRRK, sequence number 58), PYC36L-TAT (GRKKRRQRRRGGLQGRRRQGYQSIKP, sequence number 59), R6W3 based on penetratin (RRWWRRWRR, sequence number 60), R9-hLF (RRRRRRRRRKCFQWQRNMRKVRGPPVSCIKR, sequence number 61), RA9 (RRAARRARR, sequence number 62), RH9(RRHHRRHRR, sequence number 63), RL9(RRLLRRLRR, sequence number 64), RVG-9R (YTIWMPENPRPGTPCDIFTNSRGKRASNG-9R, sequence number 65), RW9 (RRWWRRWRR, sequence number 66), S8 (SSSSSSSSK, sequence number 67), SAP (VRLPPPVRLPPPVRLPPP, sequence number 68), SAPr (PPPLRVPPPLRVPPPLRV, sequence number 69), SynB1 (RGGRLSYSRRRFSTSTGR, sequence number 70), SynB3 (RRLSYSRRRF, sequence number 71), Xentry (LCLRPVG, sequence number 72), YARA (YARAAARQARAKALARQLGVAA, sequence number 73), TAT (47-57) (GRKKRRQRRRP, sequence number 74), TAT (48-60) (GRKKRRQRRRPPQ, sequence number 75), TAT (49-57) (RKKRRQRRR, sequence number 76), TAT-D (GRRRQRRKKRG, sequence number 77), TAT-L (GRKKRRQRRRG, sequence number 78), TAT-PEG-Cholesterol (YGRKKRRQRRR-PEG-cholesterol, sequence number 79), TP-10 (AGYLLGKINLKALAALAKKIL, sequence number 80), TP-10K (AGYLLGKINKLKALAALAKKIL, sequence number 81), transportan (GWTLNSAGYLLGKINLKALAALAKKIL, sequence number 82), CAD-2 (GLWRALWRLLRSLWRLLWKA-NH-CH₂-CH²-SH, sequence number 83), PF6 (AGYLLGK(K(K2(TFQ4)))INLKALAALAKKIL, sequence number 84), and polyarginine derivative (RRRQRRKRRRQ, sequence number 85).

Among these cell-penetrating peptides, from the viewpoint of affinity with a cell membrane and ease of synthesis, a cell-penetrating peptide formed of an amino acid sequence that is rich in basic amino acid residues such as arginine, lycine, histidine and triptophan (for example, half or more of the total amino acid residues are basic amino acid residues) is preferred. Examples of such cell-penetrating peptides include oligoarginine (Rn, n is the number of arginine residues ranging from 6 to 12) and TAT derived from a Tat protein of human immunodeficiency virus 1 (HIV-1). A cell-penetrating peptide formed of an amino acid sequence that is rich in basic amino acid residues is considered to induce macropinocytosis, a form of endocytosis that is a process of a cell to take in extracellular substance. As a result, it is considered that the centrally-acting peptide derivative is introduced into a cell in a more efficient manner.

From the viewpoint of the efficiency of cell penetration of the centrally-acting peptide derivative, the cell-penetrating peptide is preferably a peptide in which half or more of the total amino acid residues are arginine residues, more preferably oligoarginine formed of 6 to 12 arginine residues, further preferably oligoarginine formed of 7 to 9 arginine residues, yet further preferably oligoarginine formed of 8 arginine residues.

### (Endosomal-escape Portion)

The centrally-acting peptide derivative has an endosomal-escape portion. An endosomal-escape portion is considered to shorten the time for the centrally-acting peptide derivative to remain in a cell, and enables escape from the endosome in a shorter period. As a result, it is considered to become possible to expedite the delivery of the centrally-acting peptide derivative to the brain and the distribution of the centrally-acting peptide derivative in the brain.

The structure or the mechanism of endosomal escape of the endosomal-escape portion is not specifically limited, as long as it has an activity of promoting the escape of the centrally-acting peptide derivative from an endosome. Examples of the endosomal-escape portion include an acetyl group (CₙH₂ₙ₊₁CO—, n=3˜9) and a penetration accelerating sequence (PAS) such as FFLIPKG (sequence number 86), LILIG (sequence number 87), FFG (sequence number 88), FFFFG (sequence number 89) and FFFFFFG (sequence number 90). From the viewpoint of the function of causing endosomal escape, a PAS is a preferred endosomal escape portion.

The position of the cell-penetrating sequence portion and the endosomal-escape portion in the centrally-acting peptide derivative is not particularly restricted. For example, the cell-penetrating sequence portion may be positioned closer to the centrally-acting portion than the endosomal-escape portion, or the endosomal-escape portion may be positioned closer to the centrally-acting portion than the cell-penetrating sequence portion. From the viewpoint of achieving the effect of the invention more efficiently, the centrally-acting peptide derivative preferably has the cell-penetrating sequence portion at a position closer to the centrally-acting portion than the endosomal-escape portion, and more preferably has a PAS as the endosomal-escape portion at the N-terminal side of the cell-penetrating sequence portion.

### (Spacer)

In the centrally-acting peptide derivative, the centrally-acting portion may be linked to the cell-penetrating sequence portion or the endosomal-escape portion directly or via a spacer. When a spacer is positioned between the centrally-acting portion and the cell-penetrating sequence portion, the amino acid sequence of the spacer is not particularly restricted as long as it has a function to suppress a decrease or a loss of the activity of the centrally-acting portion. Generally, the cell-penetrating sequence portion is formed of basic amino acid residues. Therefore, when the centrally-acting portion includes an acidic amino acid residue, it is preferred to position a spacer having a sequence formed of one or more (for example, 1 to 10, preferably 2 to 6) neutral amino acid residues, such as glycine, in order to suppress a decrease or a loss of the activity of the centrally-acting portion caused by an interaction between the cell-penetrating sequence portion and the centrally-acting portion.

The centrally-acting peptide derivative may be subjected various modification according to the intended purposes. Examples of the modification include amino group modification (such as biotinylation, myristoylation, palmitoylation, acetylation and maleimidation), carboxy group modification (such as amidation and esterification), thiol group modification (such as farnesylation, geranylation, methylation and palmitoylation), hydroxy group modification (such as phosphorylation, sulfation, octanoylation, palmitoylation and palmitoleoylation, fluorescence labelling (such as FITC, FAM, rhodamine, BODIPY, NBD and MCA), pegylation, and introduciton of unnatural amino acids, D-amino acids or the like. The modification may be performed on any of the centrally-acting portion, cell-penetrating sequence portion, the endosomal-escape portion or the spacer of the centrally-acting peptide derivative.

Each of the amino acid residues that constitute the centrally-acting peptide derivative may be either L-form or D-form, as long as the effect of the invention is achieved. The method of obtaining the centrally-acting peptide derivative is not particularly restricted, and may be extracted from a living body or a natural substance, or may be prepared by a genetic engineering process or an organic synthetic process.

In an embodiment, the centrally-acting peptide derivative is formed of an amino acid sequence in which an amino acid sequence of PAS, an amino acid sequence of CPP, an amino acid sequence of a spacer as necessary, and an amino acid sequence of the centrally-acting portion are positioned in this order from the N-terminal side, where PAS is selected from FFLIPKG, LILIG, FFG, FFFFG or FFFFFFG, CPP is selected from oligoarginine (Rn, n=6−12), and the spacer is selected from glycine residues (Gn, n=2−6).

When the centrally-acting portion of the centrally-acting peptide derivative is an amino acid sequence, the total number of amino acid residues of the centrally-acting peptide derivative is not particularly restricted as long as the effect of the invention is achieved, and may be selected according to the intended purposes. For example, the total number of amino acid residues of the centrally-acting peptide derivative may be 80 or less, preferably 70 or less, more preferably 60 or less. For example, the total number of amino acid residues of the centrally-acting peptide derivative may be 35 or more, preferably 40 or more, more preferably 45 or more.

### <Pharmaceutical Composition>

The pharmaceutical composition of the invention includes a centrally-acting peptide derivative having a centrally-acting portion, a cell-penetrating sequence portion and an endosomal-escape portion as an active ingredient. By including a centrally-acting peptide derivative as an active ingredient, the pharmaceutical composition can be rapidly delivered to the brain and efficiently exert a pharmacological effect. Further, since the pharmaceutical composition can be administered is a less invasive manner, it is useful as a drug that needs to be taken on a daily basis at home. Therefore, a nasal formulation is a preferred form of the pharmaceutical composition.

The neuropsychiatric disorder, which is a therapeutic objective of the pharmaceutical composition, is not particularly restricted as long as a therapeutic effect is exerted by the action of the centrally-acting portion of the centrally-acting peptide derivative on the central nervous system. Examples of the neuropsychiatric disorder include depression, learning disorder, anxiety, eating disorder, cognition disorder, high blood pressure, sleeping disorder and epilepsia.

Specific examples of the pharmaceutical composition include an anti-depression agent, a learning disorder-alleviating agent, an anti-anxiety agent, a feeding suppression agent, a cognitive disorder-alleviating agent, a blood-pressure-lowering agent, an analgesic agent, a sleep-inducing agent, and an anti-epileptic agent. The type of the centrally-acting portion, the cell-penetrating sequence portion and the endosomal-escape portion of the centrally-acting peptide derivative may be selected according to the therapeutic objective. For example, a pharmaceutical composition including a centrally-acting peptide derivative having a peptide portion derived from GLP-2 as an active ingredient is effective as an anti-depression agent. Further, since GLP-2 exerts a blood pressure-lowering activity, the pharmaceutical composition including a centrally-acting peptide derivative having a peptide portion derived from GLP-2 as an active ingredient is considered to be particularly effective for a patient with depression due to strong pressure and high blood pressure in combination. A pharmaceutical composition including a centrally-acting peptide derivative having a peptide portion derived from GLP-1 or NmU as an active ingredient is effective as a learning disorder-alleviating agent.

The details and the preferred embodiments of the centrally-acting peptide derivative included in the pharmaceutical composition are as described above.

The method of using the pharmaceutical composition is not particularly restricted, and may be any of intranasal administration, oral administration, intravenous administration and the like. In view of adaptability to daily administration, intranasal administration and oral administration are preferred. In view of adaptability to delivery to the brain, intranasal administration is more preferred. The pharmaceutical composition may include a component other than the centrally-acting peptide derivative. Examples of the component other than the centrally-acting peptide derivative include a medium and a formulation additive that are used for the preparation of a pharmaceutical composition. Specific examples of the formulation additive include a diluent, a disintegrant, a binder, a lubricant, a surfactant, a buffer, a solubilizing agent, a stabilizer, a tonicity agent, a suspending agent, an emulcifier, a solvent, a thickner, a mucolytic agent, a humectant and a preservative. The dosage amount of the pharmaceutical composition may be selected according to the type of disease, the symptomatic state, weight or age of the patient, the administration form and the like.

### <Nasal Formulation>

The nasal formulation of the invention includes a centrally-acting peptide derivative having a centrally-acting portion, a cell-penetrating sequence portion and an endosomal-escape portion as an active ingredient. By including a centrally-acting peptide derivative as an active ingredient, the nasal formulation can be rapidly delivered to the brain and efficiently exert a pharmacological effect. Further, since the nasal formulation can be administered is a less invasive manner, it is useful as a drug that needs to be taken on a daily basis at home. Therefore, a nasal formulation is a preferred form of the pharmaceutical composition.

The details and the preferred embodiments of the centrally-acting peptide derivative included in the nasal formulation are as described above. The nasal formulation may include a component other than the centrally-acting peptide derivative. Examples of the component other than the centrally-acting peptide derivative include a medium and a formulation additive that may be used for the preparation of the pharmaceutical composition as descried above.

The embodiments of the invention include a use of the pharmaceutical composition including a centrally-acting peptide derivative as an active ingredient, as described above, for intranasal administration. The details and the preferred embodiments of the centrally-acting peptide derivative for the use are as described above.

The embodiments of the invention include a method of stabilizing a centrally-acting peptide, the method including adding a cell-penetrating sequence portion and an endosomal-escape portion to the centrally-acting peptide. The details and the preferred embodiments of the cell-penetrating sequence portion, the endosomal-escape portion and the centrally-acting peptide in the method are as described above.

### EXAMPLES

The invention are explained in further detail by referring to the following examples. The materials, amounts, rates, procedures and the like may be modified without departing from the scope of the invention. Therefore, the scope of the invention should not be construed in a restricted manner by the examples as described below.

Evaluation on anti-depression activity of centrally-acting peptide peptide derivative

### Example 1

A derivative of GLP-2 (GLP-2 derivative) having an amino acid sequence of FITC-FFLIPKG-(R)₈-(G)₂-HADGSFSDEMNTILDNLAARDFINWLIQTKITD was prepared by an ordinary method as a centrally-acting peptide derivative. The GLP-2 derivative was primarily dissolved in DMSO and diluted with phosphate-buffered saline (PBS) to adjust the final concentration of DMSO to 16%, thereby preparing a DMSO solution of GLP-2 derivative.

The anti-depression activity of the GLP-2 derivative was evaluated by a forced-swim test on rats using the DMSO solution of GLP-2 derivative. The forced-swim test was performed in accordance with the following procedure.

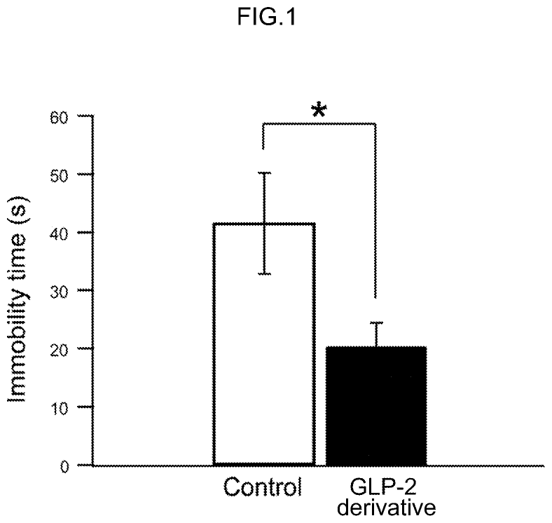

One hour before the test, rats (8-10 week old, male, n=6) were transferred to the laboratory for habituation. The rats were intranasally administered with the DMSO solution of GLP-2 derivative by 18 μg/day for two days, 30 minutes before the test. The rats were allowed to swim for 15 minutes, one by one in a transparent plastic cylinder with a diameter of 18 cm and a height of 50 cm filled with water of 25±1° C. to a height of 30 cm. During the swimming, the immobility time (s) was measured. The rats after the test were wiped with a Kimtowel and placed back to a cage. The test was performed for consecutive 2 days, day 1 as a preliminary test and day 2 as a main test. As a control, rats (8-10 week old, male, n=6) were intranasally administered with a solvent without GLP-2 derivative by 10 μL and subjected to a forced-swim test in the same manner. The results are shown in Table 1 and FIG. 1. The error bar represents mean±SE (n=6), and the statistical processing was performed by Mann-Whitney test (*P<0.05).

### Comparative Example 1

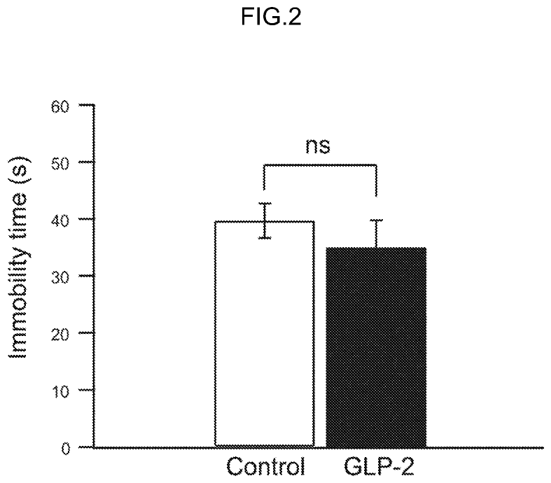

A peptide having an amino acid sequence of HADGSFSDEMNTILDNLAARDFINWLIQTKITD, i.e., an amino acid sequence of GLP-2 alone without a cell-penetrating sequence portion and an endosomal-escape portion, was prepared by an ordinary method, and a DMSO solution of GLP-2 (16%) was prepared in the same manner as Example 1. The forced-swim test was performed in the same manner as Example 1, except that the rats (8-10 week old, male, n=6) were intranasally administered with the DMSO solution of GLP-2 by 18 μg/day. The results are shown in Table 1 and FIG. 2. The error bar represents mean±SE (n=6), and the statistical processing was performed by Mann-Whitney test (ns refers to not significant).

TABLE 1
Immobility time (s)

| Example 1 | | Comparative Example 1 | |
|---|---|---|---|
| Control | GLP-2 derivative | Control | GLP-2 |
| 55 | 36 | 48 | 45 |
| 22 | 13 | 43 | 41 |
| 45 | 8 | 35 | 30 |
| 25 | 25 | 47 | 33 |
| 76 | 16 | 30 | 46 |
| 26 | 24 | 35 | 15 |

### Example 2

The state of distribution of GLP-2 derivative in olfactory bulb, infralimbic cortex, basolateral amygdala, central amygdala), medial amygdala, paraventricular hypothalamic nucleus, dorsomedial hypothalamic nucleus, hippocampus and parabrachial nucleus of rats was evaluated by the following process. The rats were intranasally administered with a DMSO solution of GLP-2 derivative (16%) by 18 μg in the same manner as Example 1.

Thirty minutes after the intranasal administration to a rat with a DMSO solution of GLP-2 derivative (16%) as prepared in the same manner as Example 1, the rat was formalin-fixed to prepare a brain specimen. A section (30 μm) was obtained from the brain specimen with a cryostat (Leica Microsystems M1560S), and the section was observed with a fluorescence microscope (Keyence Corporation, BZ-9000). As a control, a brain specimen of a rat intranasally administered with a solvent without GLP-2 was prepared and observed in the same manner. The results are shown in Table 1. The error bar represents mean±SE (n=6), and the statistical processing was performed by Mann-Whitney test (*P<0.05, **P<0.01).

### Example 3

A GLP-2 derivative having an amino acid sequence of FFLIPKG-(R)₈-(G)₂-HADGSFSDEMNTILDNLAARDFINWLIQTKITD was prepared as a centrally-acting peptide derivative. This DLP-2 derivative is different from the GLP-2 derivative prepared in Example 1 in that FITC is not added thereto. The GLP-2 derivative is primarily dissolved in DMSO and diluted with phosphate buffer (PBS) in order to adjust the final concentration of DMSO to 16%, thereby preparing a DMSO solution of GLP-2 derivative.

The anti-depression activity of the GLP-2 derivative was evaluated by a forced-swim test on a mouse. The test was performed by the following process.

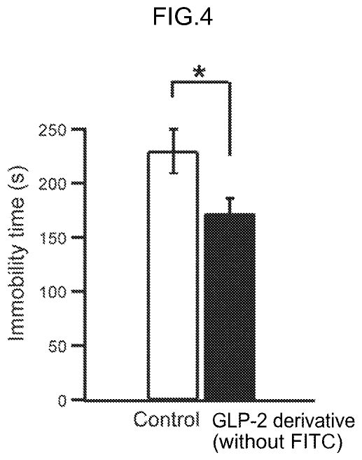

Twenty-four hours before the test, ddY mice (5-7 week old, male, n=12) were subjected to a preliminary swimming for 15 minutes. The mice were administered GLP-2 derivative by 3.6 μg with the DMSO solution, on the day before the test and on the day for the test, respectively. Then, the test was performed in the same manner as Example 1, and the immobility time (s) was measured. As a control, mice (5-7 week old, male, n=12) were administered with a solution without GLP-2 derivative were subjected to a forced-swim test in the same manner. The results are shown in Table 2 and FIG. 4. The error bar represents mean±SE (n=12), and the statistical processing was performed by Mann-Whitney test (*P<0.05).

TABLE 2
lmmobility time (s)

| Control | GLP-2 derivative |
|---|---|
| 205 | 163 |
| 263 | 65 |
| 216 | 217 |
| 218 | 147 |
| 278 | 185 |
| 47 | 152 |
| 276 | 154 |
| 175 | 151 |
| 239 | 231 |
| 280 | 218 |
| 321 | 236 |
| 236 | 141 |

### Example 4

The anti-depression activity of the GLP-2 derivative was evaluated by a tail suspension test on a mouse, using the DMSO solution of GLP-2 derivative as prepared in Example 3. The test was performed by the following process.

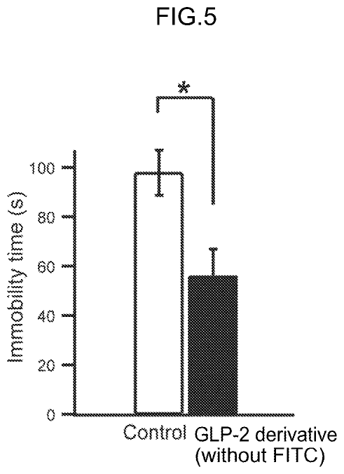

On the day before the test and on the day for the test, ddY mice (5-7 week old, male, n=9) were administered with the GLP-derivative by 3.6 μg, respectively, with the DMSO solution. Then, the mice were suspended by the tail and the immobility time (s) was measured. As a control, mice (5-7 week old, male, n=9) were intranasally administered with a solvent without GLP-2 derivative and subjected to a tail suspension test in the same manner. The results are shown in Table 3 and FIG. 5. The error bar represents mean±SE (n=9), and the statistical processing was performed by Mann-Whitney test (*P<0.05).

TABLE 3
Immobility time (s)

| Control | GLP-2 derivative |
|---|---|
| 57 | 27 |
| 74 | 49 |
| 121 | 131 |
| 147 | 45 |
| 73 | 71 |
| 101 | 34 |
| 107 | 27 |
| 92 | 54 |
| 109 | 67 |

### Example 5

The anti-depression activity of the GLP-2 derivative was evaluated by a forced-swim test on a depression model rat using the DMSO solution of GLP-2 derivative as prepared in Example 3.

As the depression model mouse, ddY mice (5-7 week old, male, n=11) were administered with adrenocorticotropic hormone (ACTH) to induce an enhanced state of hypothalamic-pituitary-adrenal axis (HPA system). The administration of ACTH was performed by 0.45 mg/kg/day for consecutive 14 days.

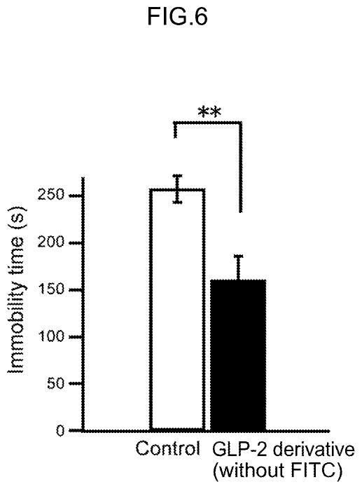

The forced-swim test was performed in the same manner as Example 3 and the immobility time (s) was measured. The mice were intranasally administered with GLP-2 derivative by 3.6 μg as a DMSO solution of GLP-2 derivative, each day for consecutive 6 days until the day for the test. As a control, mice (5-7 week old, male, n=11) were administered with a solvent without GLP-2 derivative by 4 μL each day for consecutive 6 days and subjected to a forced-swim test in the same manner. The results are shown in Table 4 and FIG. 6. The error bar represents mean±SE (n=11), and the statistical processing was performed by Mann-Whitney test (*P<0.01).

TABLE 4
Immobility time (s)

| Control | GLP-2 derivative |
|---|---|
| 193 | 25 |
| 214 | 113 |
| 251 | 268 |
| 307 | 149 |
| 177 | 287 |
| 265 | 58 |
| 289 | 207 |
| 310 | 192 |
| 252 | 151 |
| 316 | 221 |
| 264 | 96 |

Evaluation on learning disorder-alleviating activity of centrally-acting peptide derivative

### Example 6

A derivative of GLP-1 (7-36 amide) having an amino acid sequence of FFLIPKG-(R)₈-(G)₂-HAEGTFTSDVSSYLEGQAAKEFIAWLVKGR-NH₂ was prepared by an ordinary method as a centrally-acting peptide derivative. The GLP-1 derivative was primarily dissolved in FOS 12 (n-dodecylphosphocholine), and diluted with PBS to adjust the final concentration of FOS 12 to 1%, thereby preparing a FOS 12 solution of GLP-1 derivative.

The learning disorder-alleviating activity of the GLP-1 derivative was evaluated by a Y-maze test on a learning disorder-induced mouse, using the FOS 12 solution of GLP-1 derivative.

As the learning disorder-induced mouse, ddY mice (5-7 week old, male, n=5) were administered with lipopolysaccharide (LPS) by 10 μg at three days before the test. Fifteen minutes before the administration of LPS, the mice were administered with GLP-1 derivative by 3 μg with the FOS 12 solution of GLP-1 derivative.

The Y-maze test was performed by using a device having three arms of the same size. The number of times that the mouse entered the different arm for consecutive three times was counted, and the result was divided by a number obtained by deducting 2 from the total number of times of entering the arm. The result was multiplied by 100, and used as a spontaneous alternation behavior (%).

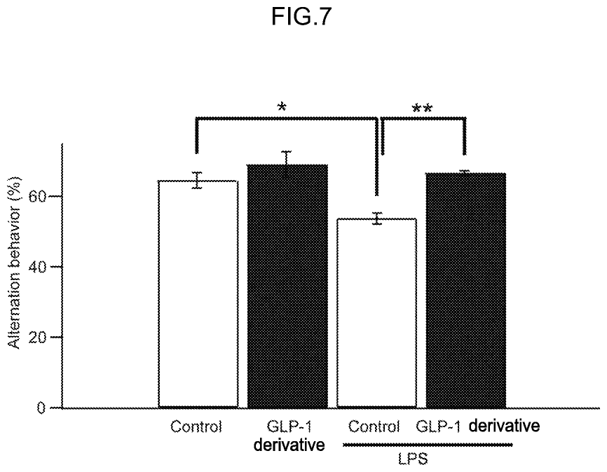

As a control, mice (5-7 week old, male, n=5 or 7), intranasally administered a solvent without GLP-1 derivative by 4 μL 15 minutes before the administration of LPS, were subjected to the same test as the above. Further, mice that were not administered with LPS (vehicle) were subjected to the same test as the above. The results are shown in Table 5 and FIG. 7. The error bar represents mean±SE (n=5 or 7), and the statistical processing was performed by Mann-Whitney test (*P<0.05, **P<0.01).

TABLE 5
Alternation behavior (%)

| Vehicle | | LPS | |
|---|---|---|---|
| Control | GLP-1 derivative | Control | GLP-1 derivative |
| 65.7 | 72.2 | 52.0 | 66.7 |
| 60.0 | 70.3 | 56.5 | 66.7 |
| 66.7 | 78.6 | 48.6 | 67.9 |
| 59.6 | 67.6 | 54.8 | 67.4 |
| 61.5 | 56.4 | 56.8 | 64.3 |
| 76.0 | | | |
| 62.1 | | | |

Evaluation on learning disorder-alleviating activity of centrally-acting peptide derivative

### Example 7

A derivative of NmU having an amino acid sequence of FFFFG-(R)₈-(G)₂-YKVNEYQGPVAPSGGFFLFRPRN-NH₂ was prepared by an ordinary method as a centrally-acting peptide derivative. The NmU derivative was primarily dissolved in FOS 12 and diluted with PBS to adjust the final concentration of FOS 12 to 5%, thereby preparing a FOS 12 solution of NmU derivative.

As the learning disorder-induced mouse, ddY mice (5-7 week old, male, n=6) administered with lipopolysaccharide (LPS) by 10 μg three days before the test were used. Fifteen minutes before the administration of LPS, the mice were administered with NmU derivative by 3 μg with the FOS 12 solution of NmU derivative. A Y-maze test was performed in the same manner as Example 6, and a spontaneous alternation behavior (%) was calculated from the measurement results.

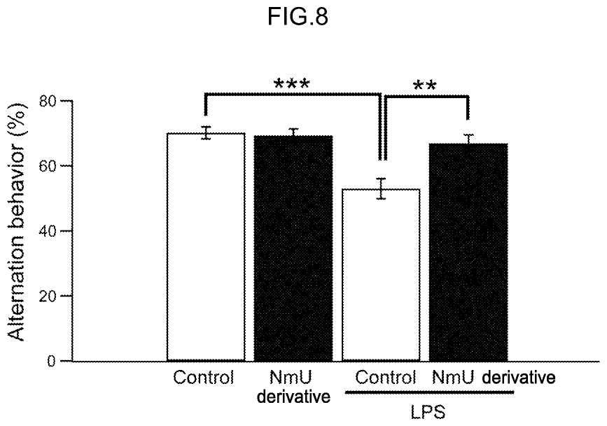

As a control, mice (5-7 week old, male, n=6), which were intranasally administered a solvent without NmU derivative by 4 μL 15 minutes before the administration of LPS, were subjected to the same test as the above. Further, mice not administered with LPS (vehicle) were subjected to the same test as the above. The results are shown in Table 6 and FIG. 8. The error bar represents mean±SE (n=6), and the statistical processing was performed by Mann-Whitney test (*P<0.01, **P<0.001).

TABLE 6
Alternation behavior (%)

| Vehicle | | LPS | |
|---|---|---|---|
| Control | NmU derivative | Control | NmU derivative |
| 67.7 | 69.0 | 68.2 | 66.7 |
| 66.7 | 63.9 | 52.1 | 60.0 |
| 79.1 | 67.8 | 50.8 | 74.1 |
| 69.2 | 78.8 | 48.9 | 69.4 |
| 68.6 | 65.9 | 49.0 | 58.6 |
| 69.6 | 70.5 | 48.9 | 72.4 |

Evaluation on stability of centrally-acting peptide derivative

### Example 8

The GLP-2 and the GLP-2 derivative prepared in Example 3 were primarily dissolved in DMSO and diluted with PBS to adjust the final concentration of DMSO to 1%, respectively, thereby preparing samples.

The content of GLP-2 or GLP-2 derivative in each sample was adjusted to 12 ng/μL, and DPP-4 (dipeptidyl peptidase-4) was added to each sample.

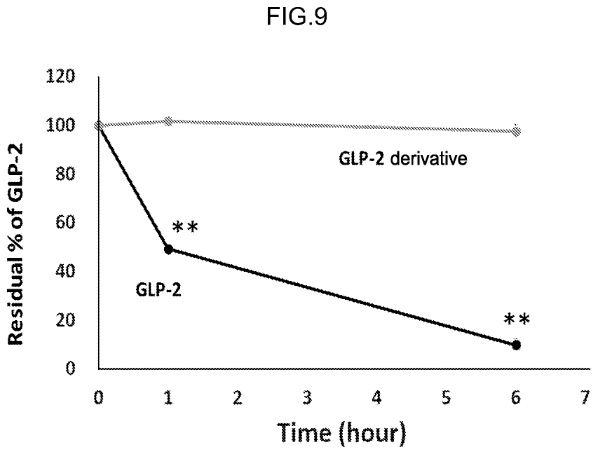

The samples were stored at 37° C., and the content of the GLP-2 or GLP-2 derivative was measured at 1 hour and 6 hours after the preparation. The measured values were converted to a relative value as a residual ratio (%) based on the content of the GLP-2 or GLP-2 derivative immediately after the preparation as 100%. The results are shown in FIG. 9. The value shown in FIG. 9 represents mean±SE (n=3), and the statistical processing was performed by t-test (**P<0.001).

### Analysis on Evaluation Results

The results of Example 1 and Comparative Example 1 showed that the immobility time in the forced-swim test of the group administered with GLP-2 derivative, prepared by adding a cell-penetrating sequence portion and an endosome-escape portion to GLP-2, was significantly shorter than that of the control group, indicating a significant difference in the anti-depression activity between the groups. In contrast, no significant difference in the anti-depression activity was shown between the group administered with GLP-2, not added with a cell-penetrating sequence portion and an endosome-escape portion, and the control group. These results suggest that the anti-depression activity of GLP-2 is improved by adding a cell-penetrating sequence portion and an endosome-escape portion thereto.

The results of Example 2 show that the GLP-2 derivative widely distributes in the brain, indicating that the improvement in anti-depression activity, achieved by adding a cell-penetrating sequence portion and an endosomal-escape portion to GLP-2, is due to the enhanced distribution of GLP-2 in the brain.

The results of Example 3 and Example 4, in which the test object and the test method were changed, and Example 5, in which a treatment-resistive depression model was used, suggest that an effect of suppressing depression is obtained by the administration of GLP-2.

The results of Example 6 and Example 7, in which a Y-maze test was performed with GLP-1 and NmU as a centrally-acting peptide, suggest that the learning ability is significantly ameliorated by the administration of GLP-1 or NmU, while the learning ability of the LPS-administered group was lower than that of the vehicle group (not administered with LPS).

The results of Example 8, a stability test of a centrally-acting peptide derivative, showed that the GLP-2 derivative is more stable than GLP-2 alone with respect to DPP-4, which is an enzyme capable of decomposing GLP-2. These results suggest that the addition of a cell-penetrating sequence portion and an endosomal-escape portion to a centrally-acting portion suppress the decomposition of GLP-2 by DPP-4, thereby providing additional effects such as improved stability of a peptide and improved ability of migrating to the central nervous system.

The disclosure of Japanese Patent Application Nos. 2014-177974 and 2014-184436 is incorporated herein in its entirety. All publications, patent applications, and technical standards mentioned in this specification are herein incorporated by reference to the same extent as if each individual publication, patent application, or technical standard was specifically and individually indicated to be incorporated by reference.

1. A method of delivering a centrally-acting peptide derivative to a central nervous system by intranasal administration, the centrally-acting peptide derivative comprising a centrally-acting portion, a cell-penetrating sequence portion, and an endosomal-escape portion.

2. The method according to claim 1, wherein the centrally-acting portion is an amino acid sequence portion that is derived from a peptide.

3. The method according to claim 2, wherein the peptide is selected from the group consisting of GLP-1, GLP-2, neuromedin U, an opioid peptide, oxytocin, leptin, orexin, neuropeptide Y, and insulin.

4. The method according to claim 1, wherein the cell-penetrating sequence portion is an oligoarginine represented by Rn where n is from 6 to 12.

5. The method according to claim 1, wherein the endosomal-escape portion is FFLIPKG or FFFFG.

6. The method according to claim 1, wherein the centrally-acting portion is an amino acid sequence portion derived from a peptide selected from the group consisting of GLP-1, GLP-2, and neuromedin U, the cell-penetrating sequence portion is an oligoarginine represented by Rn where n is from 6 to 12, and the endosomal-escape portion is FFLIPKG or FFFFG.

7. The method according to claim 1, wherein the number of amino acid residues of the peptide is 35 or more and 70 or less.

8. The method according to claim 1, used for treatment of a neuropsychiatric disorder.

9. The method according to claim 1, used for treatment of depression or a learning disorder.

10. The method according to claim 1, wherein the centrally-acting peptide derivative is delivered by axonal transport.

11. A method of treating a neuropsychiatric disorder, the method comprising delivering a centrally-acting peptide derivative to a central nervous system by intranasal administration, the centrally-acting peptide derivative comprising a centrally-acting portion, a cell-penetrating sequence portion, and an endosomal-escape portion.

12. The method according to claim 11, wherein the centrally-acting portion is an amino acid sequence portion that is derived from a peptide.

13. The method according to claim 12, wherein the peptide is selected from the group consisting of GLP-1, GLP-2, neuromedin U, an opioid peptide, oxytocin, leptin, orexin, neuropeptide Y, and insulin.

14. The method according to claim 11, wherein the cell-penetrating sequence portion is an oligoarginine represented by Rn where n is from 6 to 12.

15. The method according to claim 11, wherein the endosomal-escape portion is FFLIPKG or FFFFG.

16. The method according to claim 11, wherein the centrally-acting portion is an amino acid sequence derived from a peptide selected from the group consisting of GLP-1, GLP-2 and neuromedin U, the cell-penetrating sequence portion is an oligoarginine represented by Rn where n is from 6 to 12, and the endosomal-escape portion is FFLIPKG or FFFFG.

17. The method according to claim 11, wherein the number of amino acid residues of the peptide is 35 or more and 70 or less.

18. The method according to claim 11, used for treatment of depression or a learning disorder.

19. The method according to claim 11, wherein the centrally-acting peptide derivative is delivered by axonal transport.


SEQUENCE LISTING (90 sequences)

SEQ ID NO: 1 - protein, 33 aa, Homo sapiens
  MISC_FEATURE: GLP-2
HADGSFSDEMNTILDNLAARDFINWLIQTKITD

SEQ ID NO: 2 - protein, 30 aa, Homo sapiens
  MISC_FEATURE: GLP-1 (7-36)amide
HAEGTFTSDVSSYLEGQAAKEFIAWLVKGR

SEQ ID NO: 3 - protein, 23 aa, Unknown
  Porcine Spial Cord
  MISC_FEATURE: Neuromedin U
YKVNEYQGPVAPSGGFFLFRPRN

SEQ ID NO: 4 - protein, 19 aa, Artificial sequence
  Angiopep-5
RFFYGGSRGKRNNFRTEEY

SEQ ID NO: 5 - protein, 16 aa, Artificial sequence
  Antp
RQIKIWFQNRRMKWKK

SEQ ID NO: 6 - protein, 11 aa, Artificial sequence
  Antp 6 Bac
YGRKKRRQRRR

SEQ ID NO: 7 - protein, 13 aa, Artificial sequence
  BR1
RAGLQFPVGRLLR

SEQ ID NO: 8 - protein, 17 aa, Artificial sequence
  BR2
RAGLQFPVGRLLRRLLR

SEQ ID NO: 9 - protein, 21 aa, Artificial sequence
  Buf IIb[BR3]
RAGLQFPVGRLLRRLLRRLLR

SEQ ID NO: 10 - protein, 5 aa, Artificial sequence
  SH-CPPP-2
KLPVM

SEQ ID NO: 11 - protein, 10 aa, Artificial sequence
  CyLoP-1
CRWRWKCCKK

SEQ ID NO: 12 - protein, 17 aa, Artificial sequence
  Cys-Antp
CRQIKIWFQNRRMKWKK

SEQ ID NO: 13 - protein, 19 aa, Artificial sequence
  Cys-pVEC
CLLIILRRRIRKQAHAHKS

SEQ ID NO: 14 - protein, 19 aa, Artificial sequence
  Cys-SAP
CVRLPPPVRLPPPVRLPPP

SEQ ID NO: 15 - protein, 19 aa, Artificial sequence
  Cys-SAPr
CPPPLRVPPPLRVPPPLRV

SEQ ID NO: 16 - protein, 12 aa, Artificial sequence
  Cys-TAT
CYGRKKRRQRRR

SEQ ID NO: 17 - protein, 22 aa, Artificial sequence
  Cys-TP10
CAGYLLGKINLKALAALAKKIL

SEQ ID NO: 18 - protein, 23 aa, Artificial sequence
  Cys-TP10K
CAGYLLGKINKLKALAALAKKIL

SEQ ID NO: 19 - protein, 21 aa, Artificial sequence
  FAK
FAKLAARLYRKALARQLGVAA

SEQ ID NO: 20 - protein, 9 aa, Artificial sequence
  H9
HHHHHHHHH

SEQ ID NO: 21 - protein, 15 aa, Artificial sequence
  HC-[poly(K)]
QYIKANSKFIGITEL

SEQ ID NO: 22 - protein, 22 aa, Artificial sequence
  hLF
KCFQWQRNMRKVRGPPVSCIKR

SEQ ID NO: 23 - protein, 22 aa, Artificial sequence
  HR9
CHHHHHHRRRRRRRRRHHHHHC

SEQ ID NO: 24 - protein, 32 aa, Artificial sequence
  IR9
GLFEAIEGFIENGWEGMIDGWYGRRRRRRRRR

SEQ ID NO: 25 - protein, 33 aa, Artificial sequence
  JNKI 1D TAT
TDQSRPVQPFLNLTTPRKPRRPPRRRQRRKKRG

SEQ ID NO: 26 - protein, 9 aa, Artificial sequence
  K9
KKKKKKKKK

SEQ ID NO: 27 - protein, 14 aa, Artificial sequence
  Low molecular weight protamine (LMWP)
VSRRRRRRGGRRRR

SEQ ID NO: 28 - protein, 18 aa, Artificial sequence
  MAP
KLALKLALKALKAALKLA

SEQ ID NO: 29 - protein, 14 aa, Artificial Sequence
  Mitoparan
  MISC_FEATURE (10)..(10): Aib - 2-Aminoisobutyric aic
INLKKLAKLXKKIL

SEQ ID NO: 30 - protein, 25 aa, Artificial sequence
  m-N2-10-1 (MTS-C)
HYSRLEICNLAAVALLPAVLLALLP

SEQ ID NO: 31 - protein, 25 aa, Artificial sequence
  m-N2-10-1 (MTS-N)
AAVALLPAVLLALLPHYSRLEICNL

SEQ ID NO: 32 - protein, 23 aa, Artificial sequence
  m-N2-10-1 (NIV-C)
HYSRLEICNLGRKKRRQRRRPPQ

SEQ ID NO: 33 - protein, 23 aa, Artificial sequence
  m-N2-10-1 (NIV-N)
GRKKRRQRRRPPQHYSRLEICNL

SEQ ID NO: 34 - protein, 18 aa, Artificial sequence
  m-N2-10-1 (OR-C)
RRRRRRRRHYSRLEICNL

SEQ ID NO: 35 - protein, 18 aa, Artificial sequence
  m-N2-10-1 (OR-N)
HYSRLEICNLRRRRRRRR

SEQ ID NO: 36 - protein, 31 aa, Artificial sequence
  MPG-8
AFLGWLGAWGTMGWSPKKKRKCYSTEAMIDE

SEQ ID NO: 37 - protein, 32 aa, Artificial sequence
  MPG
GALFLAAALSLMGLWSQPKKKRKVNHCHCHSH

SEQ ID NO: 38 - protein, 32 aa, Artificial sequence
  MPG
GALFLGAALSLMGLWSQPKKKRKVNHCHCHSH

SEQ ID NO: 39 - protein, 14 aa, Artificial sequence
  NrTP1
YKQCHKKGGKKGSG

SEQ ID NO: 40 - protein, 15 aa, Artificial Sequence
  NrTP2
  MISC_FEATURE (10)..(10): Ahx- Aminohexanoic Acid
YKQCHKKGGXKKGSG

SEQ ID NO: 41 - protein, 14 aa, Artificial sequence
  NrTP5
YKGCHKKGGKKGSG

SEQ ID NO: 42 - protein, 14 aa, Artificial sequence
  NrTP6
YKQSHKKGGKKGSG

SEQ ID NO: 43 - protein, 14 aa, Artificial sequence
  NrTP7
YRQSHRRGGRRGSG

SEQ ID NO: 44 - protein, 27 aa, Artificial sequence
  MPG HIV-gp41/SV40 T-antigen
GALFLGFLGAAGSTMGAWSQPKKKRKV

SEQ ID NO: 45 - protein, 14 aa, Artificial sequence
  NrTP8
WKQSHKKGGKKGSG

SEQ ID NO: 46 - protein, 19 aa, Artificial sequence
  PAMAM-PEG-Angiopep-2
PFFYGGSGGNRNNYLREEY

SEQ ID NO: 47 - protein, 12 aa, Artificial sequence
  PEI-TAT
TGRKKRRQRRRP

SEQ ID NO: 48 - protein, 16 aa, Artificial sequence
  Penetratin (43-58)
RQIKIWFQNRRMKWKK

SEQ ID NO: 49 - protein, 17 aa, Artificial sequence
  Penetratin
RQIKIWFQNRRMKWKKK

SEQ ID NO: 50 - protein, 21 aa, Artificial sequence
  Pep-1 HIV-reverse transcriptase/SV40 T-antigen
KETWWETWWTEWSQPKKKRKV

SEQ ID NO: 51 - protein, 21 aa, Artificial sequence
  Pep-1
KETWWETWWTEWSQPKKKRKV

SEQ ID NO: 52 - protein, 28 aa, Artificial sequence
  pepM residues 45-72 of DENV-2 C protein
KLFMALVAFLRFLTIPPTAGILKRWGTI

SEQ ID NO: 53 - protein, 35 aa, Artificial sequence
  pepR residues 67-100 of DENV-2 C protein
LKRWGTIKKSKAINVLRGFRKEIGRMLNILNRRRR

SEQ ID NO: 54 - protein, 13 aa, Artificial sequence
  peptide derived from protamine
RRRRRRGGRRRRG

SEQ ID NO: 55 - protein, 18 aa, Artificial sequence
  Peptide vascular endothelial cadherin (pVEC)
LLIILRRRIRKQAHAHSK

SEQ ID NO: 56 - protein, 21 aa, Artificial sequence
  PF14
  misc_feature (10)..(11): Xaa can be any naturally occurring amino acid
  misc_feature (18)..(19): Xaa can be any naturally occurring amino acid
AGYLLGKLLXXLAAAALXXLL

SEQ ID NO: 57 - protein, 16 aa, Artificial sequence
  PR9
FFLIPKGRRRRRRRRR

SEQ ID NO: 58 - protein, 15 aa, Artificial sequence
  protamine
VSRRRRRRGGRRRRK

SEQ ID NO: 59 - protein, 26 aa, Artificial sequence
  PYC36L-TAT
GRKKRRQRRRGGLQGRRRQGYQSIKP

SEQ ID NO: 60 - protein, 9 aa, Artificial sequence
  R6W3 Based on penetratin
RRWWRRWRR

SEQ ID NO: 61 - protein, 31 aa, Artificial sequence
  R9-hLF
RRRRRRRRRKCFQWQRNMRKVRGPPVSCIKR

SEQ ID NO: 62 - protein, 9 aa, Artificial sequence
  RA9
RRAARRARR

SEQ ID NO: 63 - protein, 9 aa, Artificial sequence
  RH9
RRHHRRHRR

SEQ ID NO: 64 - protein, 9 aa, Artificial sequence
  RL9
RRLLRRLRR

SEQ ID NO: 65 - protein, 38 aa, Artificial sequence
  RVG-9R
YTIWMPENPRPGTPCDIFTNSRGKRASNGRRRRRRRRR

SEQ ID NO: 66 - protein, 9 aa, Artificial sequence
  RW9
RRWWRRWRR

SEQ ID NO: 67 - protein, 9 aa, Artificial sequence
  S8
SSSSSSSSK

SEQ ID NO: 68 - protein, 18 aa, Artificial sequence
  SAP
VRLPPPVRLPPPVRLPPP

SEQ ID NO: 69 - protein, 18 aa, Artificial sequence
  SAPr
PPPLRVPPPLRVPPPLRV

SEQ ID NO: 70 - protein, 18 aa, Artificial sequence
  SynB1
RGGRLSYSRRRFSTSTGR

SEQ ID NO: 71 - protein, 10 aa, Artificial sequence
  SynB3
RRLSYSRRRF

SEQ ID NO: 72 - protein, 7 aa, Artificial sequence
  Xentry
LCLRPVG

SEQ ID NO: 73 - protein, 22 aa, Artificial sequence
  YARA
YARAAARQARAKALARQLGVAA

SEQ ID NO: 74 - protein, 11 aa, Artificial sequence
  TAT (47-57)
GRKKRRQRRRP

SEQ ID NO: 75 - protein, 13 aa, Artificial sequence
  TAT (48-60)
GRKKRRQRRRPPQ

SEQ ID NO: 76 - protein, 9 aa, Artificial sequence
  TAT (49-57)
RKKRRQRRR

SEQ ID NO: 77 - protein, 11 aa, Artificial sequence
  TAT-D
GRRRQRRKKRG

SEQ ID NO: 78 - protein, 11 aa, Artificial sequence
  TAT-L
GRKKRRQRRRG

SEQ ID NO: 79 - protein, 11 aa, Artificial sequence
  TAT-PEG-Cholesterol
YGRKKRRQRRR

SEQ ID NO: 80 - protein, 21 aa, Artificial sequence
  TP-10
AGYLLGKINLKALAALAKKIL

SEQ ID NO: 81 - protein, 22 aa, Artificial sequence
  TP-10K
AGYLLGKINKLKALAALAKKIL

SEQ ID NO: 82 - protein, 27 aa, Artificial sequence
  Transportan
GWTLNSAGYLLGKINLKALAALAKKIL

SEQ ID NO: 83 - protein, 20 aa, Artificial sequence
  CAD-2
GLWRALWRLLRSLWRLLWKA

SEQ ID NO: 84 - protein, 22 aa, Artificial sequence
  PF6
  misc_feature (8)..(8): Xaa can be any naturally occurring amino acid
AGYLLGKXINLKALAALAKKIL

SEQ ID NO: 85 - protein, 11 aa, Artificial sequence
  polyarginine derivative
RRRQRRKRRRQ

SEQ ID NO: 86 - protein, 7 aa, Artificial sequence
  PAS
FFLIPKG

SEQ ID NO: 87 - protein, 5 aa, Artificial sequence
  PAS
LILIG

SEQ ID NO: 88 - protein, 3 aa, Artificial sequence
  PAS
FFG

SEQ ID NO: 89 - protein, 5 aa, Artificial sequence
  PAS
FFFFG

SEQ ID NO: 90 - protein, 7 aa, Artificial sequence
  PAS
FFFFFFG